(19) United States
(12) Patent Application Publication
(10) Pub. No.: US 2002/0022805 A1
(43) Pub. Date: Feb. 21, 2002

(54) Self metering cartridge

(76) Inventor: Donovan R. Lane, Paso Robles, CA (US)

Correspondence Address: Dean P. Edmundson, P.O. Box 710, Windsor, CO, 80550, US

(21) Appl. No.: 09/828,545

(22) Filed: Apr. 7, 2001

Related U.S. Application Data
(63) Continuation-in-part of application No. 09/351,558, filed on Jul. 12, 1999, now Pat. No. 6,213,984.
(60) Provisional application No. 60/093,166, filed on Jul. 17, 1998.

(51) Int. Cl. A61M 5/32; A61M 5/00

(52) U.S. Cl. 604/210; 604/207; 604/232

(57) ABSTRACT

A system for automatically metering a predetermined dosage of a medicament from a cartridge. The system includes a tubular cartridge containing the medicament and a plunger which is movable in the cartridge to dispense the medicament through one end. The cartridge further includes an interior tab (e.g. a ring) for stopping forward movement of a detent carried by the trigger of a syringe body in which the cartridge is positioned. By limiting forward movement of the detent, forward movement of the plunger rod is also stopped, thereby controlling the amount of medicament dispensed from the cartridge.

### CROSS REFERENCE TO RELATED APPLICATION

This application is a continuation-in-part of my pending application, Ser. No. 09/351,558, filed Jul. 12, 1999, which claimed the benefit of Provisional Application No. 60/093,166, filed Jul. 17, 1998.

### BACKGROUND

1. Field of Invention

This invention relates to multidose, medical injection syringes used for the vaccination and treatment of livestock diseases. More specifically, it relates to a process and a closed handling and delivery system for those injectable animal health products used in syringes and how the dosage level administered to the patient is controlled or metered.

2. Description of Prior Art

Generally speaking, in multidose, pistol grip livestock syringes, the precise metering of the dosage dispensed is accomplished by restricting the stroke of the plunger rod. Heretofore, the stroke of the plunger rod has been controlled by mechanical systems incorporated into the syringe body and trigger mechanisms. Original designs of pistol grip syringes commonly include ratchet and pawl mechanisms, wherein the trigger is squeezed and a pawl attached to the trigger engages a ratchet, formed on the plunger rod. The plunger rod is then advanced forward to dispense the medicament from the barrel of the syringe.

Metering is accomplished by an adjustable stop attached to either the trigger, as is demonstrated in U.S. Pat. No. 3,110,310 to Cislak (1963), or to the syringe body as is shown in U.S. Pat. No. 4,014,331 to Head (1977). The adjustable stop restricts the forward movement or stroke of the plunger rod therefore metering the amount of medicament dispensed. Similar mechanisms for dosage metering are employed when pre-filled cartridges are used with pistol grip syringe bodies as is shown in U.S. Pat. No. 4,738,664 to Prindle (1988) and U.S. Pat. No. 3,517,668 to Brickson (1967). Phillips et al. (1988) in U.S. Pat. No. 4,758,233 devised a two-stage cartridge system. In this system, the dosage size is controlled by a threaded adjustment on the anterior end of the syringe body. Other known cartridge type systems generally utilize a single dose system wherein one full squeeze of the trigger dispenses the entire contents of the cartridge, as is shown in U.S. Pat. No. 4,576,591 to Kaye et al. (1986) and U.S. Pat. 4,968,303 to Clarke et al. (1990).

With these methods of dosage metering, the amount of medicament dispensed from a multi-dose cartridge is determined by a setting or an adjustment made to the syringe or applicator by the technician. Mechanical dosage settings are often bumped or may slip to the next setting so that many animals may be injected with the improper dosage before the mistake has been detected and the correction is made. It is also not uncommon for the technician to accidentally set the dosage adjustment to an improper setting. Most medicaments have a prescribed dosage level, which technically could eliminate the need for dosage adjustments by the technician.

### OBJECTS AND ADVANTAGES OF THE PRESENT INVENTION

Dosage levels for most medicaments, particularly vaccines, are constant and are prescribed by the manufacturer of the vaccine. As an example, a vaccine for the prevention of X disease may require a 5 milliliter dose while a different vaccine for Y disease may prescribe a 2 milliliter dose. In our self-metering cartridge system, all syringe bodies will be capable of dispensing any dosage level up to the largest dosage that may be prescribed. The self-metering cartridge will dictate the length of the stroke of the plunger rod therefore dictating the dosage level of the medicament dispensed with each squeeze of the trigger.

In practice, a 2-milliliter dose product will be packaged in a self-metering cartridge that will restrict the forward movement of the plunger rod and allow precisely 2 milliliters of medicament to be dispensed from the cartridge. Similarly a 5-milliliter dose self-metered cartridge will stop the forward movement of the plunger rod when 5 milliliters of medicament have been dispensed from the cartridge. If the loaded cartridge in the chamber is not equipped with the self-metering aspect, a full squeeze of the trigger will allow the plunger rod to advance as far forward as is mechanically possible. That full squeeze will dictate the largest dosage that the syringe is capable of administering. As an example, an unrestricted full squeeze may dispense 10 milliliters of medicament.

The advantage to the self-metering cartridge is that no human error or mechanical malfunction of an adjustable metering mechanism can cause the syringe to give an improper dosage. Even if the wrong medicament has been accidentally loaded into the syringe, the proper dosage for that particular medicament will be given. In addition, the stop mechanism for metering the dosage level has been simplified so that there are no adjustable or moving parts. When a cartridge is loaded into the barrel of a syringe body, the metering is automatic and specific to the product contained in the cartridge.

### BRIEF DESCRIPTION OF THE DRAWINGS

FIG. 1 is a partially cut-away view showing a pistol grip syringe body with the trigger in the resting position.

FIG. 1A is an enlargement of a portion of FIG. 1.

FIG. 2 is a partially cut-away view showing the syringe body of FIG. 1 with the trigger in the squeezed position.

FIG. 2A is an enlargement of a portion of FIG. 2.

FIG. 3 is an exploded view of the cartridge with an internal metering ring or tab.

FIG. 3A is a cross section of the cartridge metering ring or tab which is shown in FIG. 3.

FIG. 4 is a 3/4 exploded view of syringe body, cartridge and self-metering components.

### REFERENCE NUMERALS IN DRAWINGS

10a syringe body frame, right half

10b syringe body frame, left half

12 syringe body barrel

13 pin connecting syringe barrel to the frame

14 syringe body handle

15 latch for securing syringe barrel in closed position on frame

16 self metering cartridge

16a metering ring groove

18 cartridge metering ring

18a cartridge metering ring lock rib

20 trigger

20a lower portion of the trigger

20b upper portion of the trigger

22 trigger pivot

24 moveable metering rod

26 metering rod hinge point

28 plunger rod

30 drag link

32 plunger

34 cartridge seal

36 standard rubber stopper

38 standard aluminum stopper seal

### DETAILED DESCRIPTION OF THE INVENTION

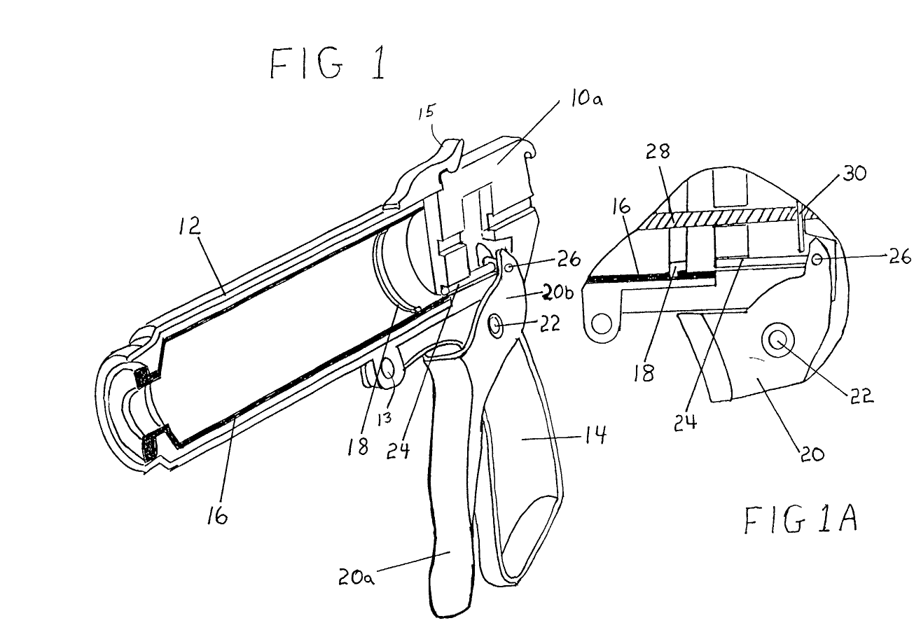

The essence of this invention combines the mechanical aspects of the items in FIGS. 1 through 4 to complement the systems described in U.S. Pat. No. 5,964,736 and application Ser. No. 08/918,733, incorporated herein by reference. The goals of this self-metering cartridge are as follows:

(a) Simplify the mechanism to automatically meter the dosage given by this syringe and cartridge system.

(b) Eliminate the need to preset or adjust the syringe to meter the dosage to be given.

(c) Eliminate the chance of human or mechanical error often associated with mechanical hand adjustments of the syringe to meter the dosage levels.

(d) Transfer the responsibility of dosage metering from the syringe body to the cartridge. Therefore a medicament will be packaged into a self-metering cartridge, preset to the dosage level prescribed by the manufacturer of the medicament. No adjustments need to be or can be made by the technician administering the medicament to the livestock; therefore, the proper dosage level is always given.

(e) Reduce the cost to manufacture the metering device in the syringe.

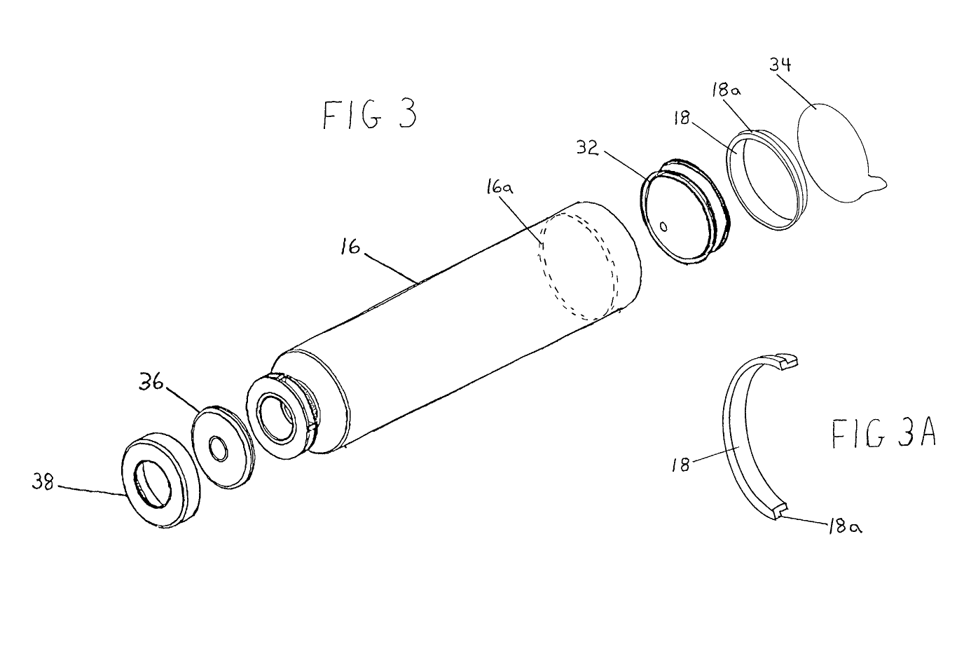

A preferred embodiment of the self-metering cartridge is illustrated in FIGS. 1 through 4. The disposable, self-metering cartridge 16 as shown in FIG. 3 is a cylindrical hollow tube made of a durable or unbreakable, plastic-like substance. The anterior end is necked down to form a standard vaccine-type bottle-filling head. The filling head is plugged with a standard rubber-like stopper 36 and sealed with a standard aluminum stopper seal 38. The medicament is sealed into the cartridge 16 at the posterior end by the plunger 32. The cartridge metering tab or ring 18 is installed posterior to the plunger 32 and a seal 34 is applied to the extreme posterior end of the cartridge 16. The cartridge 16 is molded with an interior metering ring groove 16a to accept the cartridge metering ring lock rib 18a of the cartridge metering ring 18 when inserted and snapped into place in the cartridge 16 during assembly. FIG. 3A is a cross section of the cartridge metering ring and displays the configuration of the cartridge metering ring lock rib 18a. The placement of the metering ring 18 in the cartridge 16 dictates the distance the trigger 20 may travel with one full squeeze of the lower trigger portion 20a, which, in turn, dictates the extent of forward movement of the plunger rod 28. Forward movement of the upper trigger portion 20b, is transferred to the plunger rod 28 by the drag link 30. The distance the plunger rod 28 may travel dictates the dosage level of medicament dispensed from the cartridge 16. The moveable metering rod 24 is hinged at point 26 on the upper trigger portion 20b and transfers the movement of the upper trigger portion 20b, to the cartridge metering ring 18. When the moveable metering rod 24 strikes the cartridge metering ring 18 the forward movement of the upper trigger portion 20b and the plunger rod 28 are halted, thus determining the dosage level.

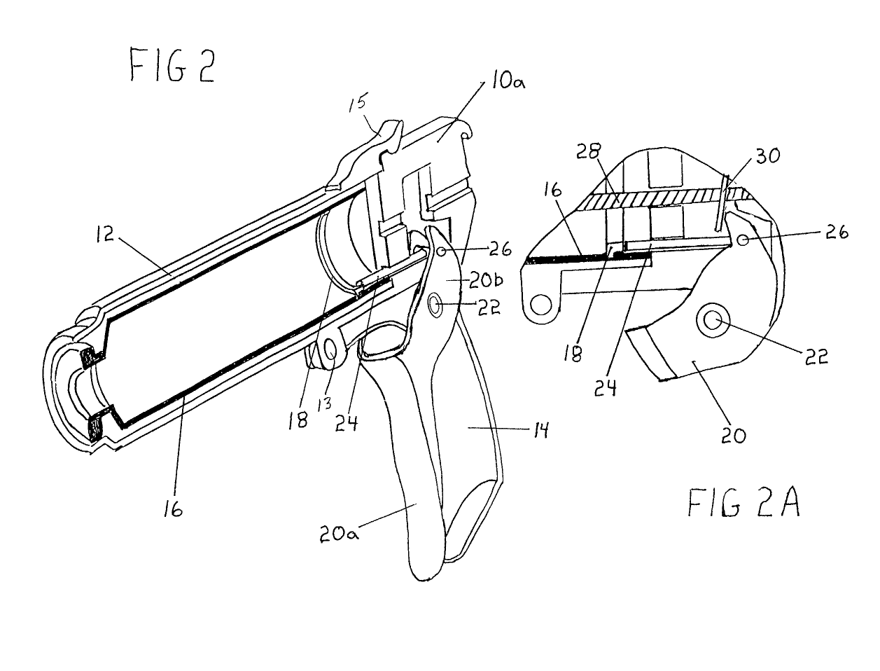

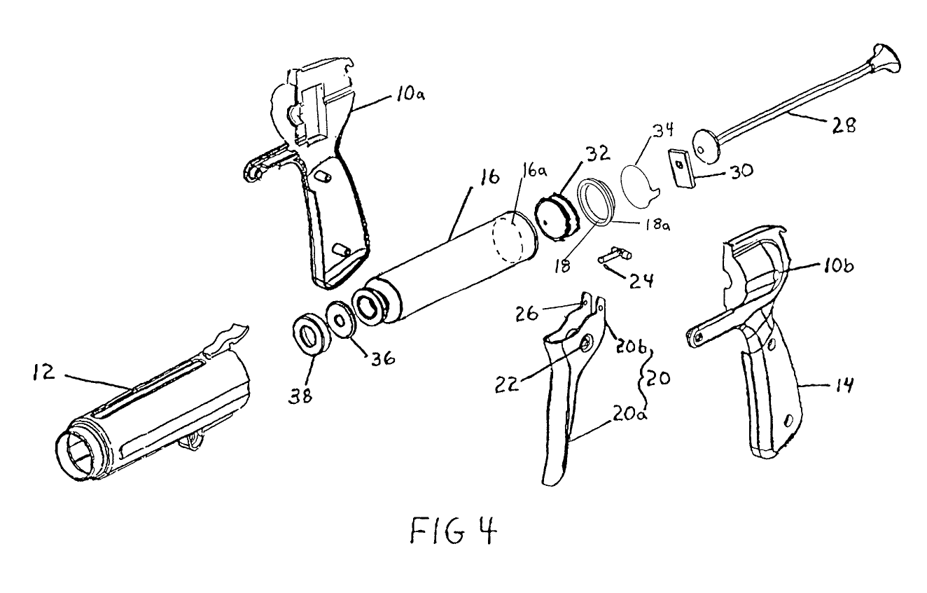

FIG. 1 and 1A demonstrate the position of the trigger 20, the drag link 30 and the moveable metering rod 24, all in the resting position. FIG. 2 and 2A demonstrate the position of those components in the compressed position i.e. with the lower trigger portion 20a pulled rearwardly toward the syringe body handle 14. FIG. 4 is an exploded view of the syringe body, cartridge and self metering components which serves to illustrate the design of those major components associated with the function of the self-metering cartridge.

### Operation of Structures of FIGS. 1 to 4

The seal 34 is removed from the posterior end of the cartridge 16 and the prefilled self-metering cartridge 16 is breech loaded into the syringe body barrel 12 (which is pivotally mounted to the gun frame by pin 13) and snapped to the closed position to the main syringe body frame 10a and 10b (where it is secured with latch 15). To administer a dose of medicament the lower trigger portion 20a is squeezed or compressed to the syringe body handle 14. As the trigger 20 rotates on the trigger pivot 22, the upper trigger portion 20b moves forward, pushing the drag link 30 forward. The forward angular movement of the drag link 30 creates a bind on the plunger rod 28 and drags the plunger rod 28 forward. The forward movement of the plunger rod 28 pushes the plunger 32 forward in the cartridge 16, forcing the medicament from the cartridge 16 through an adapted needle, pierced into the stopper 36 at the anterior end of the cartridge 16. The moveable metering rod 24 is hinged to the upper trigger portion 20b at the metering rod hinge point 26. When the trigger 20 is compressed, the moveable metering rod 24 is advanced forward until it makes contact with the cartridge metering ring 18. This contact stops the forward movement of the entire trigger mechanism including the moveable metering rod 24, the trigger 20, the drag link 30, the plunger rod 28 and the plunger 32. Thus the positioning of the metering ring 18 along the longitudinal axis of the cartridge 16 determines the travel distance of the plunger rod 28 and dictates the dosage level of medicament dispensed from the self-metering cartridge 16. The metering rod 24 serves as a detent means for stopping forward movement of the plunger 32 when rod 24 strikes ring 18 in the cartridge 16. Other types of detents could be used, if desired.

Other types of tabs could be used in the interior of the cartridge to limit the travel of the metering rod 24. A continuous ring 18 as shown and described herein is preferred as the tab means because it eliminates the need to angularly align the cartridge with the position of the metering rod 24. The tab means may be a separate element which is attached or secured to the interior wall of the cartridge, or it may be integrally formed on the interior wall of the cartridge (e.g. during molding of the cartridge).

### Summary, Ramifications and Scope

The use of this self-metering cartridge is a tremendous improvement over the current state of the art dosage metering mechanisms available in today's pistol grip syringes. The advantages listed below become apparent to anyone who has given injections to livestock.

No complicated adjustable metering mechanisms need to be incorporated into the construction of the syringe body.

There is no chance of the metering adjustment skipping or being bumped into a different setting.

The possibility of the technician accidentally dialing an improper setting on a metering adjustment is eliminated.

The dosage level for the medicament is preset and automatic, and incorporated into the cartridge by the manufacturer of the medicament. Therefore the manufacturer can rest assured that the proper dosage levels of their products are being administered.

Farm and ranch managers can also rest assured that their employees are administering the proper dosage levels of these expensive medicaments.

Although some example specifications are implied throughout the text of the above descriptions, these should not be construed as limiting the scope of the invention but as merely providing illustrations so that the reader may visualize the embodiment of the invention. Thus the scope of the invention should be determined by the appended claims and their legal equivalents, rather than by the examples given.

What is claimed is:

1. A system for automatically metering a predetermined dosage of a medicament from a cartridge, the system comprising:
(a) an elongated tubular cartridge containing said medicament, the cartridge including anterior and posterior ends and a plunger moveable in said cartridge to dispense said medicament through said anterior end; wherein said cartridge further includes tab means in the interior thereof;
(b) a pistol grip syringe body capable of receiving said cartridge and including a trigger mounted on said body; said trigger being moveable;
(c) an elongated plunger rod carried by said syringe body and having a forward end axially aligned with said cartridge; and
(d) detent means moveable by said trigger for stopping forward movement of said plunger rod when said detent means contacts said tab means in said cartridge.

2. A system in accordance with claim 1, wherein said tab means comprises a ring secured to said interior of said cartridge.

3. An elongated tubular cartridge for containing a medicament, said cartridge including anterior and posterior ends and a plunger positioned within said cartridge; wherein said plunger is moveable in said cartridge to dispense said medicament through said anterior end; wherein said cartridge further comprises an interior wall with tab means projecting into said cartridge from said interior wall; wherein said tab means is spaced a predetermined distance from said posterior end.

4. A cartridge in accordance with claim 3, wherein said tab means comprises a ring projecting inwardly into said cartridge.

5. A pistol grip syringe body dispensing member including a main frame and further comprising:
(a) a tubular barrel portion sized for receiving and supporting an elongated cartridge;
(b) a trigger moveably mounted on said frame and being moveable between open and retracted positions;
(c) an elongated plunger rod having a forward end; wherein said plunger rod is axially aligned with said cartridge when positioned in said barrel portion;
(d) detent means being moveable by said trigger and being adapted to engage tab means in the interior of a cartridge positioned in said barrel portion to thereby stop forward movement of said plunger rod at a predetermined point;
wherein movement of said trigger from said open position to said retracted position causes said plunger rod to advance said plunger rod a predetermined distance.
